(19) United States
(12) Patent Application Publication
(10) Pub. No.: US 2018/0256661 A1
(43) Pub. Date: Sep. 13, 2018

(54) Pharmaceutical Composition for Prevention and Treatment of Flatulence and Diarrhea in Rabbit and Formula and Application Thereof

(71) Applicants: Sichuan Xingzhi Zhihui Intellectual Property Operation Co., Ltd., Chengdu, Sichuan (CN); Pengxi Falian Agricultural and Livestock Co., Ltd., Suining, Sichuan (CN)

(72) Inventor: Anquan Huang, Suining (CN)

(21) Appl. No.: 15/545,724

(22) PCT Filed: May 11, 2016
(86) PCT No.: PCT/CN2016/081707

§ 371 Date: Jul. 24, 2017

(30) Foreign Application Priority Data
Dec. 1, 2015 (CN) 201510865032.2

(51) Int. Cl. A61K 36/284; A61P 1/12; A61P 1/14; A61K 36/14; A61K 36/31; A61K 36/232; A61K 36/73; A61K 36/882

(52) U.S. Cl. CPC A61K 36/284; A61P 1/12; A61P 1/14; A61K 36/882; A61K 36/31; A61K 36/232; A61K 36/73; A61K 36/14

(57) ABSTRACT

Pharmaceutical composition and formula thereof for prevention and treatment of flatulence and diarrhea in rabbits comprise 1-5 parts by weight of Rhizoma atractylodis, 1-5 parts by weight of cypress fruit, 0.5-2 parts by weight of fried radish seed, 1-3 parts by weight of Angelica dahurica, 1-3 parts by weight of Herba schizonepetae, 3-5 parts by weight of herb of shiny cinquefoil, and 1-2 parts by weight of grassleaf sweetflag rhizome. A pharmaceutical formula, wherein the active ingredients thereof are the aforementioned pharmaceutical composition. The present invention can prevent and treat the flatulence and diarrhea in rabbits effectively, induce resuscitation, and promote digestion to eliminate food stagnation. The present invention is low in costs and has significant therapeutic effect, with the cure rate of flatulence and diarrhea in rabbits being higher than 95%.

### FIELD OF THE INVENTION

The present invention relates to the technical field of health raising of rabbits, in particular to a pharmaceutical composition for prevention and treatment of flatulence and diarrhea in rabbits.

### BACKGROUND OF THE INVENTION

Rabbit flatulence is resulted from gases produced from fermentation of ingested vegetables or poor digestion caused by lacking of exercises. Rabbit flatulence often occurs in young rabbits for that body functions of young rabbits after weaning have yet fully developed, and in absence of the supply of active substances from breast milk, pH value in the gastrointestinal tract decreases, resulting in corresponding decline in activities of endocrine digestive enzymes. As a result, the speed of secretion of digestive enzymes is unable to meet the demand of digestion, weakening peristalsis of the cecum, thereby prolonging the time for which food stays in the cecum and significantly increasing the activity of harmful intestinal bacteria. Consequently, the harmful bacteria prevent poorly indigested food from being reabsorbed effectively in the colon, and the indigested food is subsequently defecated by intestinal peristalsis, which clinically manifests as diarrhea. The aforementioned is the causes of flatulence and diarrhea in rabbits.

At present, flatulence and diarrhea in rabbits is still a difficult issue to solve, and no effective clinical solution has yet existed.

### BRIEF SUMMARY OF THE INVENTION

The present invention provides a Chinese pharmaceutical substrate and a pharmaceutical composition thereof for prevention and treatment of flatulence and diarrhea in rabbits. The pharmaceutical composition is demonstrated to prevent and treat rabbit flatulence and diarrhea effectively, induce resuscitation, and promote digestion to eliminate food stagnation.

To solve the above technical problems, the present invention employs the following technical solutions:

A Chinese pharmaceutical substrate for prevention and treatment of flatulence and diarrhea in rabbits includes 1-5 parts by weight of Rhizoma atractylodis, 1-5 parts by weight of cypress fruit, and 0.5-2 parts by weight of fried radish seed.

Preferably, the Chinese pharmaceutical substrate includes 1-2 parts by weight of Rhizoma atractylodis, 1-2 parts by weight of cypress fruit, and 0.5-1 parts by weight of fried radish seed.

A pharmaceutical composition for prevention and treatment of flatulence and diarrhea in rabbits includes the aforementioned pharmaceutical substrate and further includes 1-3 parts by weight of Angelica dahurica, 1-3 parts by weight of Herba schizonepetae, 3-5 parts by weight of herb of shiny cinquefoil, and 1-2 parts by weight of grassleaf sweetflag rhizome.

Preferably, the pharmaceutical composition includes 1-2 parts by weight of Angelica dahurica, 1 part by weight of Herba schizonepetae, 3-5 parts by weight of herb of shiny cinquefoil, and 1 part by weight of grassleaf sweetflag rhizome.

A pharmaceutical composition for prevention and treatment of flatulence and diarrhea in rabbits includes the aforementioned pharmaceutical substrate and further includes 3-5 parts by weight of Radix aucklandiae, 2-6 parts by weight of Magnolia officinalis, 2-3 parts by weight of Citrus aurantium, 1-2 parts by weight of rhubarb, 3-5 parts by weight of hawthorn fruit, and 2-3 parts by weight of massa medicata fermentata.

Preferably, the pharmaceutical composition includes 3 parts by weight of Radix aucklandiae, 2 parts by weight of Magnolia officinalis, 2 parts by weight of Citrus aurantium, 1 part by weight of rhubarb, 3 parts by weight of hawthorn fruit, and 3 parts by weight of massa medicata fermentata.

Rhizoma atractylodis is used to treat damp retention in the spleen and stomach, excess disorder of the stomach, diarrhea, edema, weak foot and flaccidity disease, rheumatic arthralgia, anemofrigid cold, night blindness, and dizzy or dry eyes. As recorded in “New Compilation of Materia Medica,” Rhizoma atractylodis may dry the stomach, strengthen the spleen, and promote sweatiness to eliminate damp. Rhizoma atractylodis may also ascend and effuse Yang-Qi in the stomach, stop vomiting and diarrhea, and dispel phlegm.

Cypress fruit has the functions of expelling wind-evil, relieving uneasiness, cooling blood and stopping bleeding. Thus, cypress fruit may be used to treat influenza, headache, fever, stomachache, irritation and hematemesis.

Fried radish seed is used to suppress Qi and stop asthma, promote digestion, and reduce phlegm. Therefore, fried radish seed may treat cough, asthma due to excessive phlegm, indigestion, Qi stagnation, chest distress, flatulence, diarrhea and tenesmus. The present invention employs the aforementioned Chinese traditional medicinal ingredients to stabilize gastrointestinal functions, guide Qi, cool blood and relive uneasiness in rabbits, so as to strengthening the total body wellness of rabbits. Meanwhile, other Chinese traditional medicinal ingredients are supplemented to guide Qi, stop diarrhea, and harmonize gastrointestinal functions of rabbits to achieve the treatment of the fundamental causes of the disorders.

A pharmaceutical formula, wherein the active ingredients thereof are the aforementioned pharmaceutical composition.

The pharmaceutical formula is in the forms of granule, tablet, capsule, or pill. The content of active ingredients contained therein is 10-90 wt %, with the remaining being pharmaceutical adjuvants.

An application of the aforementioned pharmaceutical formula in preparation of drugs for prevention and treatment of flatulence and diarrhea in rabbits.

As compared with the prior art, the present invention has the following beneficial effects:

1. The present invention can prevent and treat flatulence and diarrhea in rabbits effectively, induce resuscitation, and promote digestion to eliminate food stagnation.

2. The present invention is low in costs and has significant therapeutic effect, with the cure rate for flatulence and diarrhea in rabbits being higher than 95%.

### DETAILED DESCRIPTION OF THE PREFERRED EMBODIMENTS

The present invention is further illustrated as follows. It is necessary to point out that the present invention includes, but is not limited to the following specific embodiments.

### Embodiment 1

A pharmaceutical composition for prevention and treatment of flatulence and diarrhea in rabbits comprises 1 part by weight of Rhizoma atractylodis, 1 part by weight of cypress fruit, 0.5 parts by weight of fried radish seed, 1 part by weight of Angelica dahurica, 1 part by weight of Herba schizonepetae, 3 parts by weight of herb of shiny cinquefoil, and 2 parts by weight of grassleaf sweetflag rhizome.

### Embodiment 2

A pharmaceutical composition for prevention and treatment of flatulence and diarrhea in rabbits comprises 5 parts by weight of Rhizoma atractylodis, 5 parts by weight of cypress fruit, 2 parts by weight of fried radish seed, 2 parts by weight of Angelica dahurica, 1 part by weight of Herba schizonepetae, 3 parts by weight of herb of shiny cinquefoil, and 1 part by weight of grassleaf sweetflag rhizome.

### Embodiment 3

A pharmaceutical composition for prevention and treatment of flatulence and diarrhea in rabbits comprises 2 parts by weight of Rhizoma atractylodis, 2 parts by weight of cypress fruit, 0.8 parts by weight of fried radish seed, 3 parts by weight of Angelica dahurica, 2 parts by weight of Herba schizonepetae, 4 parts by weight of herb of shiny cinquefoil, and 2 by weight parts of grassleaf sweetflag rhizome.

### Embodiment 4

A pharmaceutical composition for prevention and treatment of flatulence and diarrhea in rabbits comprises 3 parts by weight of Rhizoma atractylodis, 3 parts by weight of cypress fruit, 1 part by weight of fried radish seed, 2 parts by weight of Angelica dahurica, 3 parts by weight of Herba schizonepetae, 5 parts by weight of herb of shiny cinquefoil, and 1 part by weight of grassleaf sweetflag rhizome.

### Embodiment 5

A pharmaceutical composition for prevention and treatment of flatulence and diarrhea in rabbits comprises 1 part by weight of Rhizoma atractylodis, 1 part by weight of cypress fruit, 0.5 parts by weight of fried radish seed, 3 parts by weight of Radix aucklandiae, 2 parts by weight of Magnolia officinalis, 2 parts by weight of Citrus aurantium, 1 part by weight of rhubarb, 3 parts by weight of hawthorn fruit, and 3 parts by weight of massa medicata fermentata.

### Embodiment 6

A pharmaceutical composition for prevention and treatment of flatulence and diarrhea in rabbits comprises 3 parts by weight of Rhizoma atractylodis, 3 parts by weight of cypress fruit, 1 part by weight of fried radish seed, 5 parts by weight of Radix aucklandiae, 4 parts by weight of Magnolia officinalis, 3 parts by weight of Citrus aurantium, 1 part by weight of rhubarb, 5 parts by weight of hawthorn fruit, and 2 parts by weight of massa medicata fermentata.

### Embodiment 7

A pharmaceutical composition for prevention and treatment of flatulence and diarrhea in rabbits comprises 5 parts by weight of Rhizoma atractylodis, 4 parts by weight of cypress fruit, 1.5 parts by weight of fried radish seed, 4 parts by weight of Radix aucklandiae, 5 parts by weight of Magnolia officinalis, 2 parts by weight of Citrus aurantium, 2 parts by weight of rhubarb, 4 parts by weight of hawthorn fruit, and 2 parts by weight of massa medicata fermentata.

### Embodiment 8

A pharmaceutical composition for prevention and treatment of flatulence and diarrhea in rabbits comprises 5 parts by weight of Rhizoma atractylodis, 5 parts by weight of cypress fruit, 2 parts by weight of fried radish seed, 3 parts by weight of Radix aucklandiae, 6 parts by weight of Magnolia officinalis, 2 parts by weight of Citrus aurantium, 1 part by weight of rhubarb, 3 parts by weight of hawthorn fruit, and 3 parts by weight of massa medicata fermentata.

### Embodiment 9

A pharmaceutical formula, wherein the active ingredients thereof are the pharmaceutical composition described in Embodiment 1 to Embodiment 8.

The pharmaceutical formula is in the forms of granule, tablet, capsule, or pill. The content of active ingredients contained therein is 10-90 wt %, with the remaining being pharmaceutical adjuvants.

The present invention could be carried out with reference to the contents described in the above embodiments.

The contents described above are the embodiments of the present invention. It should be understandable that the present invention is not limited by the above embodiments. The substantial modification or adjustment made by others according to the present invention still falls within the scope of the present invention.

1. A pharmaceutical substrate for prevention and treatment of flatulence and diarrhea in rabbits, comprising:
1-5 parts by weight of Rhizoma atractylodis, 1-5 parts by weight of cypress fruit, and 0.5-2 parts by weight of fried radish seed.

2. The pharmaceutical substrate for prevention and treatment of flatulence and diarrhea in rabbits according to claim 1, wherein the pharmaceutical substrate comprises:
1-2 parts by weight of Rhizoma atractylodis, 1-2 parts by weight of cypress fruit, and 0.5-1 parts by weight of fried radish seed.

3. A pharmaceutical composition for prevention and treatment of flatulence and diarrhea in rabbits, comprising:
1-5 parts by weight of Rhizoma atractylodis, 1-5 parts by weight of cypress fruit, 0.5-2 parts by weight of fried radish seed, 1-3 parts by weight of Angelica dahurica, 1-3 parts by weight of Herba schizonepetae, 3-5 parts by weight of herb of shiny cinquefoil, and 1-2 parts by weight of grassleaf sweetflag rhizome.

4. The pharmaceutical composition for prevention and treatment of flatulence and diarrhea in rabbits according to claim 3, wherein the pharmaceutical composition comprises:
1-5 parts by weight of Rhizoma atractylodis, 1-5 parts by weight of cypress fruit, 0.5-2 parts by weight of fried radish seed, 1-2 parts by weight of Angelica dahurica, 1 part by weight of Herba schizonepetae, 3-5 parts by weight of herb of shiny cinquefoil, and 1 part of grassleaf sweetflag rhizome.

5. (canceled)

6. (canceled)

7. A pharmaceutical formula, wherein active ingredients thereof are a pharmaceutical composition comprising:
1-5 parts by weight of Rhizoma atractylodis, 1-5 parts by weight of cypress fruit, 0.5-2 parts by weight of fried radish seed, 1-3 parts by weight of Angelica dahurica, 1-3 parts by weight of Herba schizonepetae, 3-5 parts by weight of herb of shiny cinquefoil, and 1-2 parts by weight of grassleaf sweetflag rhizome.

8. The pharmaceutical formula according to claim 7, wherein the pharmaceutical formula is in forms of granule, tablet, capsule, or pill; and content of the active ingredients contained therein is 10-90 wt %, with the remaining being pharmaceutical adjuvants.

9. (canceled)
